(19) United States
(12) Patent Application Publication
(10) Pub. No.: US 2002/0188300 A1
(43) Pub. Date: Dec. 12, 2002

(54) Cannula system for hard tissue implant delivery

(76) Inventors: Yves P. Arramon, Capitola, CA (US); Scott McIntyre, San Jose, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 09/876,387

(22) Filed: Jun. 6, 2001

(51) Int. Cl. A61B 17/56

(52) U.S. Cl. 606/93

(57) ABSTRACT

This is a cannula system for hard tissue implant material delivery. A cannula having at least two stages is described. A proximal section has one set of outer and inner diameter and a distal section has smaller outer and/or inner diameters. The length of each section is preferably adapted for use in performing percutaneous vertebroplasty. The cannula preferably has a section inner 13 gauge diameter and another section with an inner 11 gauge diameter. Internal and/or external transition regions may be provided between the sections of different diameters. For use in accessing a site, preferably a 13 gauge stylet is received in a lumen of the cannula. Interlocking or complimentary handle portions are preferably associated with each of the stylet and cannula.

### TECHNICAL FIELD

This relates to implements for accessing hard tissue sites and implanting tissue therein. Advantageously, they are used for performing vertebroplasty.

### BACKGROUND

The introduction of flowable material to an implantation site within a patient to effect one or more therapeutic goals is well known. The flowable material may be of high or low viscosity. Matter ranging from typical fluids or solutions to non-Newtonian fluids, pastes, gels and the like has been used for one purpose or another in the medical arts.

Various applicators have been used in order to deliver implant material. Syringes are often used for less-viscous fluids, particularly where delivery at low pressure is adequate. When higher delivery pressure is required—for instance, to handle more viscous implant material, devices such as those described in U.S. patent application Ser. No. 09/409,934, entitled “High Pressure Applicator” to Preissman, filed Sep. 30, 1999; PCT Publication No. WO 99/49819, entitled “High Pressure Applicator for Hard Tissue Implant Placement” to Preissman, filed on Mar. 26, 1999; U.S. patent application Ser. No. 09/409,948, entitled “Precision Instruments for Percutaneous Delivery of Implant Materials, to Preissman, filed Sep. 30, 1999; and U.S. patent application Ser. No. 09/408,690, entitled “High Pressure Delivery System”, to Preissman, et al., filed Sep. 30, 1999, may be required.

For effecting vertebroplasty, especially percutaneous vertebroplasty, the use of such devices to effectively drive hard tissue implant material such as Polymethylmethacrylate (PMMA) may be critical. The present invention addresses other issues associated with performing procedures involving access through hard tissue like vertebroplasty.

The general procedure for performing percutaneous vertebroplasty involves the use of a standard 11 gauge Jamshidi needle. The needle includes an 11 gauge cannula with an internal stylet. The cannula and stylet are used in conjunction to pierce the cutaneous layers of a patient above the hard tissue to be supplemented, then to penetrate the hard cortical bone of the vertebra, and finally to traverse into the softer cancellous bone underlying the cortical bone.

A large force axial force on the Jamshidi needle must be applied by a physician to drive the stylet through the cortical bone. Once penetration of the cortical bone is achieved, additional downward axial force, but at a reduced magnitude compared to that required to penetrate the cortical bone, is required to position the stylet/tip of the cannula into the required position within the cancellous bone. If the force magnitude is not reduced appropriately, or if very soft bone is encountered, as is often the case with osteoporitic patients, the stylet and cannula can be accidentally and suddenly driven through the cortical bone on the opposite side of the vertebra. This is a very dangerous and potentially lethal situation in the case of vertebroplasty, since the aorta is located in close proximity to the anterior surface of at least the thoracic and lumbar vertebrae, and could easily be punctured by such an occurrence. Additionally, with regard to all vertebrae, the spinal cord is located medially of the pedicle, and could also be damaged by a piercing stylet. Appropriate placement of a cannula to deliver implant material is shown and described in U.S. Pat. No. 6,231,615 B1, issued May 15, 2001 and PCT Publication No. WO 99/18894, filed on Oct. 13, 1998; both to Preissman and entitled “Enhanced Visibility Materials for Implantation in Hard Tissue.”

To date, some of these issues have been addressed by providing tools for a more controlled approach. As put into use by Parallax Medical, Inc. and described in U.S. Pat. No. 6,033,411, entitled “Precision Depth Guided Instruments for Use in Vertebroplasty, to Preissman, issued on Mar. 7, 2000; U.S. patent application Ser. No. 09/409,948, filed Sep. 30, 1999 and PCT Publication No. WO 99/18866 filed on Oct. 14, 1998, both to Preissman and entitled “Precision Instruments for Percutaneous Delivery of Implant Materials,” specialized stylet and cannula combinations are known. However, as will be apparent in connection with the description of the invention below, the present invention provides further improvement particularly useful for performing vertebroplasty, especially percutaneous vertebroplasty.

### SUMMARY OF THE INVENTION

The present invention includes a cannula having a section of larger external diameter and a section of smaller external diameter. For effecting vertebroplasty, the length of the smaller or decreased diameter section is preferably long enough to pass through cortical bone to access cancellous bone without the larger section or a transition region thereto penetrating cortical bone. The wall thickness of the larger and smaller outer diameter sections of the cannula may be substantially the same, but it is preferred that the smaller diameter section have a reduced wall thickness. Either way, in a preferred variation of the invention, it is contemplated that the inner diameter of the cannula decreases from one section to the next.

A smooth transition region may be provided between the at least two external diameters. Alternately, an abrupt taper or step may mark their junction. The section of decreased external and/or internal diameter should have a substantially constant cross-section. It is preferred that the section of larger diameter(s) have a substantially constant cross-section. However, it is contemplated that the larger diameter section may be tapered, stepped or otherwise configured. In instance where the internal diameter of the cannula varies from one section to the next, an internal transition region may similarly be provided.

In a most preferred variation of the invention, the cannula comprises two straight-gauge sections—one having a thin or custom wall thickness and a inner diameter complimentary with about a 13 gauge member or slightly large and one having a 11 gauge external diameter and a regular-thickness wall. The invention may comprise a cannula as such (or as otherwise described herein) in combination with a stylet and/or such other features set forth. In any case, it is to be understood that the present invention includes the devices as well as the methodology disclosed.

### BRIEF DESCRIPTION OF THE DRAWINGS

Each of the following figures diagrammatically illustrates aspects of the present invention. The illustrations provide examples of the invention. Like elements in the various figures may be represented by identical numbering. For the sake of clarity, some such numbering may be omitted.

FIG. 1A is a side view of a stylet/cannula combination according to the present invention.

FIG. 1B is a cross-sectional view of the combination in FIG. 1A taken along line A-A.

FIG. 2A is a side view of a another cannula according to the present invention.

FIG. 2B shows enlarged, sectional views of a cannula according to the present invention, the cannula having different inner diameters with internal features indicated by broken lines.

FIG. 2C shows a cross-sectional view of the cannula sections shown in FIG. 2B taken along line B-B.

FIG. 3 shows a hard tissue implantation system useable in the present invention

### DETAILED DESCRIPTION

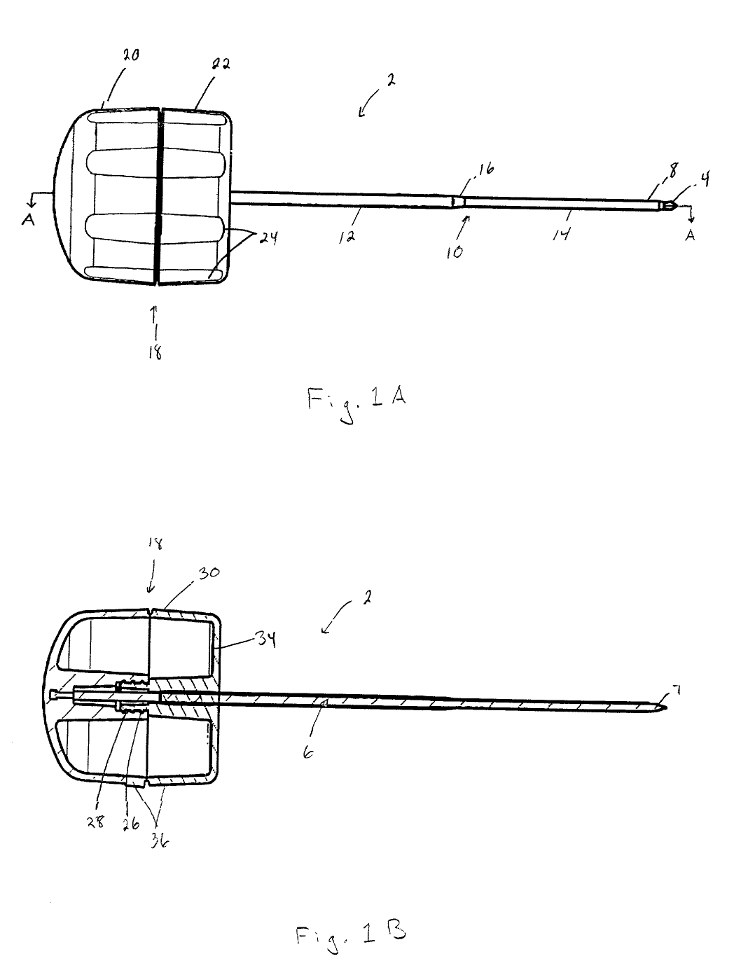

In connection with the figures, the following text provides examples or variations of the invention. Turning to FIGS. 1A and 1B, a most preferred variation of the invention is depicted. Each figure illustrates aspects of a stylet/cannula combination according to the present invention.

FIG. 1A shows the inventive toolset (2) as ready for use by a physician in driving the end (4) of stylet (6) and end (8) of cannula (10) into a hard tissue implantation site. A larger diameter proximal section (12) and a smaller diameter distal section (14) and a transition region (16) between the sections of cannula (10) is readily observable.

Preferred radial dimensions for cannula (12) are as follow. For proximal section (12), the outside diameter is preferably about 0.120 in., with a preferred wall thickness of about 0.0125 and a preferred inner diameter about 0.095 in. Such sizing corresponds to a “regualar” wall, 11 gauge section of tubing. For distal section (14), preferred dimensions include an outside diameter of about 0.090 in, with a wall thickness of about 0.0065 in. and an inner diameter of about 0.077 in. Regardless of the ultimate configuration of cannula (10) or stylet (6), stylet (6) is sized for a close fit or slip fit with at least distal portion (14) cannula (10). A preferred material for producing cannula (10) or stylet (6) is stainless steel.

While they are optimized for performing percutaneous vertebroplasty, these preferred dimensions may be varied. Particularly, the section of increased diameter may be made larger. However, the differences between the internal sizes of sections (12) and (14) may not be provided such that upon use implant material to be delivered stagnates at their junction and forms a plug blocking flow.

In order to function as an effective vertoplasty tool, distal section (14) is preferably at least 1.75 in. long. More preferably, it is about 2.0 to about 2.25 long as measured from distal end (8) to any transition section (16) so cannula (10) may be used to access most vertebrae in the human populous. The length of transition section (16) and/or proximal section (12) to handle (22) should be at least 0.5 in. for purposes of manipulating the device and providing adequate handle clearance for a handle (18) having a diameter of about 1.75 in. or greater as preferred in the present invention since cannula (10) will likely be set at an outwardly-projecting angle relative to the back of a prone patient when used for percutaneous vertebroplasty. More preferably, distal section (12)—together with or without transition section (16)—as measured up to handle (22) is about 2.5 in. long. It may be greater in length, but lengths upward of 7 to 10 in. will produce an unwieldy device.

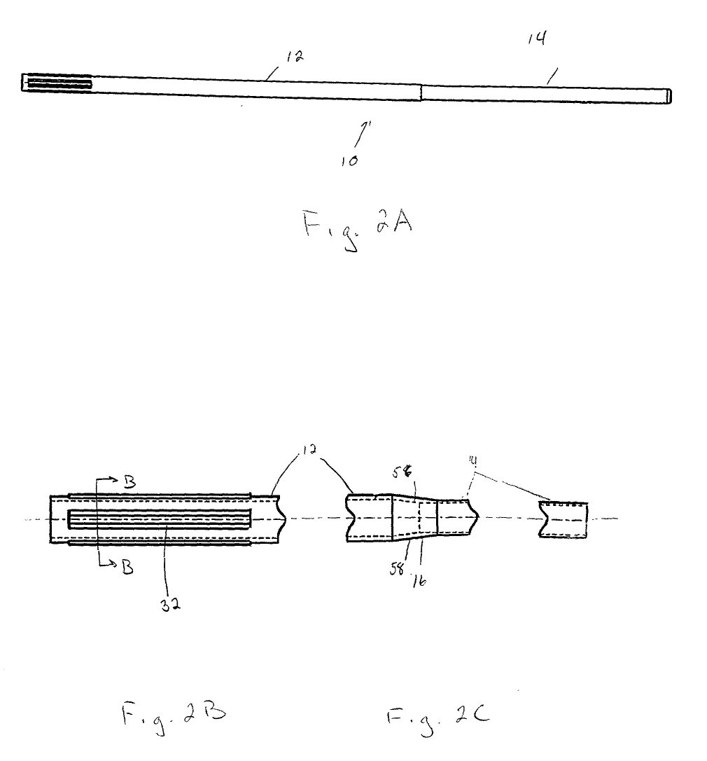

FIG. 2B illustrates preferred relative sizing of the cannula portions. FIG. 2C shows and end view of the same. Here, it may be observed that proximal section (12) is not only greater in diameter, but also has a thicker wall as compared to distal section (14). This provides strength to the design. A transition region (16) between cannula sections (12) and (14) also helps in strengthening the device. Preferably, transition region (16) includes both an internal transition (56) and an external transition (58) between the respective diameters. Their offset zones reduce stress concentration. Such a cannula is advantageously formed by swaging, though other manufacturing procedures may be employed in producing cannulas according to the present invention.

It is to be observed that a complex juncture between cannula sections (12) and (14) need not be provided. FIG. 2A shows a cannula (10) with a step between portions (12) and (14). Such a configuration is advantageously employed in a cannula having a strait-gauge lumen. A cannula so-configured is preferably produced by providing a regular wall, 11 gauge cannula and plunge grinding its distal portion to a reduced or thin wall diameter. Conventional tubing sizes and thickness designations for use in producing cannulas according to the present invention are presented at www.microgroup.com.

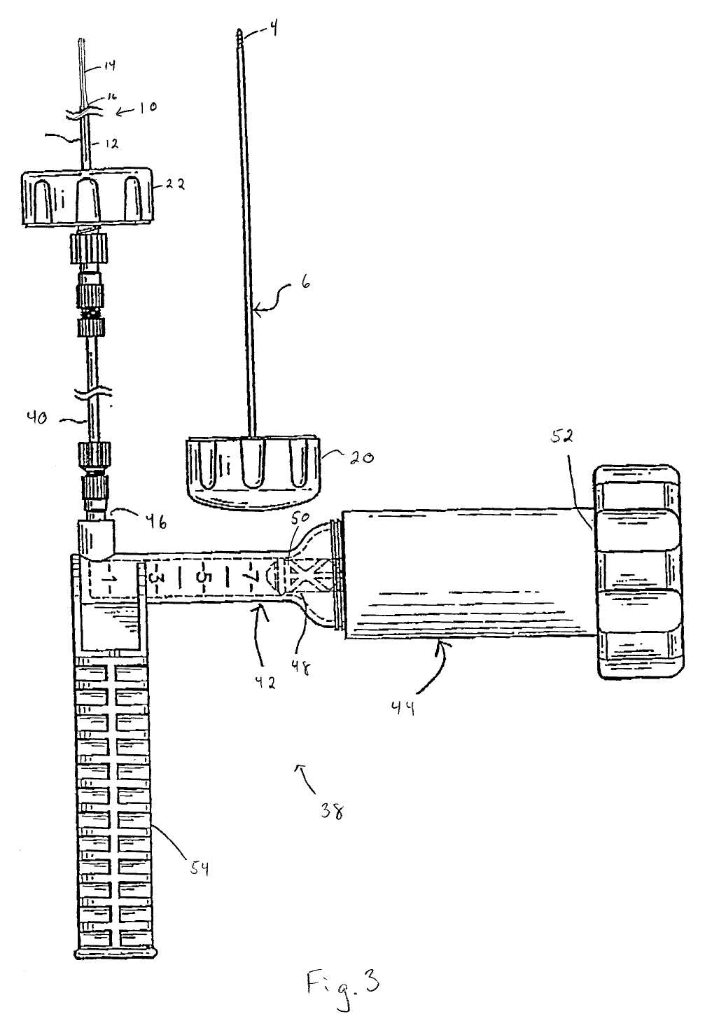

Turning now to stylet (6), as shown in FIGS. 1A, 1B and 3, it preferably comprises a straight-gauge member sized for a close fit with at least distal section (14) of cannula (10). Stylet (6) may terminate at end (4) in any of a variety of shapes. For example, a conical point, a multi-sided pointed shape (e.g. a pyramidal form), a diamond or double-beveled shape, a single-beveled shape or a threaded section may be desired. A preferred threaded section incorporates a relief groove transverse to the threads to assist in clearing material and facilitate self-tapping. Symmetrical end sections will tend to travel straight upon the application of pressure and torque as will a threaded section, while a single-beveled end section will tend to migrate in a controllable fashion, thereby allowing for some directional control. It is contemplated that, in use, a technician or a physician may wish to switch between various stylets having different end configurations to meet that challenges or needs presented in driving cannula (10) to an implantation site.

Depending on the end configuration of a given stylet (6), a combination of pressure application and torquing or screwing may be useful in accessing the implantation site. Regardless of the stylet or cannula end configuration, however, the diameter reduction of distal portion (14) of cannula (10) offered by the present invention as compared to know instruments will require the application of less force to access a site. Stated otherwise, the lessened contact area allows the application of equal or greater pressure at a lower force when driving instrument (2) through bone. The application of less force allows for more precise placement of instrument (2).

Driving and screwing procedures for the variations of the invention shown are facilitated by the provision of a handle (18) which is preferably formed by a proximal portion (20) and a distal portion (22) made of amorphous nylon—especially when the devices are to be used with PMMA due to material compatibility issues. However, other materials may be used. Preferably, the handle portions have a textured exterior surface and scallops (24) to help prevent a user's hands from slipping during use, even when the handles become wet, or the like. Screwing of the devices is performed by torquing proximal handle portion (20) or both portions (20) and (22).

Once a confirmation has been made of the proper positioning of the cannula end (8) at an implantation site, the next step is the removal of the stylet (6). Removal is performed by grasping and steadying the handle portion (20), while torquing the handle portion (22) with respect thereto. In the example shown in 1B threads (26) serve a dual purpose in fixing the cannula (10) and stylet (6) together by engaging external threads (26) with internal threads (28) of proximal handle portion (22), as well as providing part of a connection site for the source of implantable material (i.e., for linking the cannula (10) with a syringe or other tubular supply, for supplying the PMMA or other implantable material that is to be injected). Preferably, the connector formed by threads (26) is a Luer-lock type of connector, but other known connecting mechanisms may be successfully interchanged (e.g., a conventional threaded extension; a bayonet-type arrangement, etc.). The connector preferably extends above the height of the vertical wall (30) of the handle section (22) to provide easy access thereto and connection therewith.

Handle section (20) and (22) are preferably fixed to cannula (10) and stylet (6), respectively by molding thereto, although either may be alternatively or supplementally fixed by gluing, set screw(s) or other fixation means or a combination thereof. As shown in FIGS. 2A-2C in connection with cannula (10), stylet (6) may also be deformed by stamping, swedging, or other cold working, to produce features such as ribs (32) for connecting each of the stylet and cannula to its respective handle portions.

As to the configuration of handle portions, it is noted that their thin wall design, as executed in a semi-transparent material such as amorphous nylon provides certain advantages. The bottom thin wall (34) allows viewing of the site beneath the handle portion (22) at the time that the cannula is being positioned. Even the combination of the handles portions (20) and (22) may be viewed there through by using fluoroscopy or other imaging device during the placement of the stylet/cannula combination (2). When so-viewing, the additional thickness of the material of vertical walls (36) act as a border or “target” through which an operator can site and direct the cannula during the insertion process.

Although extensions to the handle portions are not shown, it is to be understood that such extensions could be provided to enhance the mechanical advantage provided to the user. Regardless, providing a decreased diameter at the cannula and stylet ends relative to that of a standard 11 gauge setup beneficially increases the mechanical advantage applied through the handle portions as shown.

An example procedure for using precision instruments according to the present invention in an intervertebral vertebroplasty will now be described. Initially, a surgeon identifies a landmark with the aid of fluoroscopy or other imaging technique. An injection is given to anesthetize the skin where insertion will occur. A long needle, having a length sufficient to percutaneously access the periosteum of the target vertebra is next used to inject anesthesia subperiostially.

After sufficient time has passed to effectively anesthetize the skin, an incision is made through the skin with a scalpel. The combined stylet and cannula (in this example, stylet (6) and cannula (10) threaded together, as shown in FIGS. 1A and 1B) are then inserted through the incision and advanced, using a translation motion with no torquing, until tip (4) of the stylet abuts the cortical bone of the vertebra or the periosteum surrounding it. Once contact has been made, the cannula tube is then grasped with a pair of hemostats and fluoroscopy/imaging is again used to assess the position of the cannula/stylet with regard to the vertebra. The hemostats are used to allow the hands of the user to be removed from the field in which the imaging radiation will be applied. With aid of the fluoroscopy/imaging view, the cannula/stylet are positioned with respect to the pedicle of the vertebra at the desired angular orientation for passing there through and into the body of the vertebra. Imaging may be performed both perpendicular to the longitudinal axis of the spine/vertebra, as well as along the longitudinal axis of the cannula/stylet, or at an angle to the longitudinal axis of the cannula stylet. As noted above, when imaging is performed along the longitudinal axis of the cannula, the handle can be used to target the tip of the instrument.

Once the orientation of the cannula/stylet has been satisfactorily set, the fluoroscopy/imaging is discontinued, the hemostats are removed, and the operator again carefully grasps the cannula/stylet being careful not to alter the orientation. Grasping the combination handle (18) and, optionally the cannula tube, the operator proceeds to both push translationally and/or torque the tool to begin advancing the stylet into the cortical bone. The devices/instruments are again viewed fluoroscopically/imaged both along the longitudinal axis of the cannula/stylet and laterally, to determine the depth of the instruments. If the desired depth has not yet been achieved, imaging is discontinued, and the cannula/stylet are forced into the cancellous bone.

Incremental advancement of the stylet/cannula with intermittent viewing by imaging is continued until the tip of the cannula has been positioned in a desirable location. Upon achieving the desired placement of the cannula, the operator grasps the handles and reverse rotates handle section (20) to remove the stylet (16) from the cannula (10), while preventing rotation of the handle (22) to leave the cannula in its position. The cannula at this stage is effectively press fit into the bone site which aids the operator in preventing rotation of the handle (22) as the handle (20) is reverse rotated. Once the stylet has been completely removed from the cannula, fluoroscopic/imaging viewing of the cannula may optionally be performed to assure that the cannula did not move during the removal of the stylet.

Next, a contrast agent (e.g., a product known as OMNIPAQUE 300 available from Nycomed in Princeton, N.J.), may be optionally injected through the cannula and the flow of the contrast agent viewed fluoroscopically or with other imaging in order to ascertain that the tip of the cannula (8) has not been placed in a vein or other significant vessel. Preferably the contrast agent is injected through injection tubing connected to the cannula, (e.g., to connection site (26)). The viewing of the flow of the contrast agent also helps to identify the shape of the vertebral body into which the injection of implant material is to be performed, as well as to locate where the major veins lie. After completing the flow of the contrast agent, the remnants of the contrast agent are flushed out of the vertebral body by injecting a flushing solution (e.g., saline) through the cannula, using a syringe or other injector. The imaging is preferably discontinued for this step. The contrast agent is flushed so that it does not occlude, cloud, or otherwise compete with the viewing of the radiopacity of the implant material when it is placed.

Next, an implant delivery device is connected to the cannula as shown in FIG. 3. For example, a high pressure applicator (38), containing implant material may be connected to the cannula (10) via a high pressure, substantially noncompliant conduit (40), as further described in U.S. patent application Ser. No. 09/688,721, entitled “Non-Compliant System for Delivery of Implant Material” to Preissman, filed on Oct. 16, 2000. An example of an effective implant material for performing vertebroplasty is a PMMA bone cement including contrast agents and/or tracer particles, aspects of which are described in U.S. Pat. No. 6,231,615 B1, issued May 15, 2001 and PCT Publication No. WO 99/18894, filed on Oct. 13, 1998; both to Preissman and entitled “Enhanced Visibility Materials for Implantation in Hard Tissue.” A detailed description of high pressure applicator (38) is provided in U.S. patent application Ser. No. 09/408,690, entitled “High Pressure Delivery System” to Preissman, et al., filed Sep. 30, 1999.

In using the pressure applicator, a tissue implant material is loaded into the first column (42) and the second column (44) is connected with the first column (42) in preparation for implantation. The introduction of air bubbles can be substantially reduced or avoided by slightly overfilling the first column and then introducing the plunger element into the material and driving the plunger into the first column to form a seal. Such action is facilitated by introduction section (48) that avoids trapping air by letting plunger end (50) interface with the bore of column (42) at a point below the implant material level.

The first column is then rotated slightly with respect to the second column until a minimal amount of tissue implant material is expressed from the fitting end (46) to ensure that no air has been entrapped in the applicator. The cannula (10) (and optional tubing 40) may be backfilled with saline, tissue implant material, or other biocompatible fluid in order to displace the air therefrom. The pressure applicator (38) is then mounted onto the cannula (10) or connected to tubing (40) that is in turn connected to cannula (10). A second purging step may be performed prior to connecting the tubing (40) to cannula (10) to both fill the tubing (40) with implant material (in cases where backfilling has been performed with something other than implant material) and as a further assurance of purging trapped air bubbles.

The operator next grasps the handle (52) in one hand and the handle (54) in the other and begins to torque the handle (52) while maintaining the handle (54) in its position. When operated as described, the pressure applicator is capable of generating pressures of about 1000 to about 4000 psi within the columns, which is a high driving force that is applied to the implantable material.

Advantageously, the pressure applicator (38) has a first column (43) which is large enough in volume (a capacitance of at least 5 cc, preferably at least 7.5 cc, and more preferably at least 10 cc and up to about 15 cc to contain sufficient implant material for an entire implantation process so that there is no need to refill the column (42) in the midst of a procedure.

In performing this procedure, whether in a vertebrae or another hard tissue implantation site, the amount and placement of implant material is tracked visually (usually through flouroscopy). When a thinner wall is used for distal section (14) making this part of cannula (10) significantly radio-translucent, a measure of flow visualization (from within the cannula) may be observed to help track flow.

However, other advantages presented by features of the present invention may be even more relevant. In comparison to a 11 gauge cannula or needle introduced to access a hard tissue implant site, the smaller diameter end of cannula (10) may be used to produce a more slender access track. This results in less tissue damage to healthy tissue. An optional bevel or transition region incorporated in the distal end (8) of cannula (10) may also be used to help reduce trauma.

Further, the slender profile of the inventive cannula allows for more precise placement and site access by applying less force on handle (18) as discussed above. This in turn allows for greater control in advancing tool (2), lessening the risk of unintended advancement and concomitant damage.

Such advantages as presented by employing a cannula with a relatively smaller diameter end often cannot, however, be realized with a simple straight gauge cannula. Conveying highly-viscous fluids through such a small diameter cannula of a length as desirously used for vertebroplasty would have to be done at extremely—perhaps prohibitively—high pressures.

Overly high implant material delivery pressures can result in certain problems. Especially with PMMA polymer/monomer mixtures that have not completely solvated, separation of the monomer from the suspended polymer has been observed. What is more, tactile feedback useful to a delivery system is lost. In turning handle (52) of applicator (38), an operator utilizing a cannula according to the present invention or a standard 11 gauge cannula can feel the resistance of implant material as it flows into a site. In contrast, in using a full-length 13 gauge cannula, the control a user would sense when having to strain to crank material out of an applicator is minimal.

Another disadvantage associated with a thin-walled straight gauge cannula in a stylet/cannula combination having a diameter about 13 Ga is that it may not sturdy enough to be pushed through cortical bone without intolerable flexure or actual failure. Instead, the present invention provides only a distal section (14) of a cannula at a critical length having a reduced diameter and/or wall thickness. In this way, buckling and flexure issues are controlled in accordance with well-know engineering principles. Put another way, a cannula “stepped down” from a stronger section to a weaker section offers better buckling strength and stiffness than a straight gauge cannula of equal length that is dimensioned like the weaker section.

Additional details as to the use or other aspects of the system described herein may be drawn from the background information cited that is intended to form part of the present invention, including any of the patent applications noted, each of which is incorporated by reference herein in its entirety for any purpose. It is noted that this invention has been described and specific examples or variations of the invention have been portrayed. The use of those specific examples is not intended to limit the invention in any way. Additionally, to the extent that there are variations of the invention which are within the spirit of the disclosure and are equivalent to features found in the claims, it is the intent that the claims cover those variations as well. All equivalents are considered to be within the scope of the claimed invention, even those which may not have been set forth herein merely for the sake of relative brevity.

Also, the various aspects of the invention described herein, in any manner or section of the application including the Abstract, Field of the Invention, Background of the Invention, Summary of the Invention, Brief Description of the Drawings, the Drawings themselves and Detailed Description, may be claimed as set forth or be modified and/or used in combination with such other aspects also described to be part of the invention either explicitly, implicitly or inherently in order to form additional variations considered to be part of the invention. Furthermore, it is contemplated that any optimal feature or any combination of optional features of the inventive variations described herein may be specifically excluded from the invention claimed and be so-described as a negative limitation.

We claim:

1. A cannula system adapted for hard tissue implantation comprising:
a tubular member defining a lumen and having a proximal and a distal end, an outside diameter of said tubular member at said proximal end being larger than an outside diameter of said tubular member at said distal end.

2. The system of claim 1, wherein said distal section has a length of between bout 0.25 inches and about 4.0 inches.

3. The system of claim 1, wherein said cannula is adapted for use in a vertebroplasty procedure.

4. The system of claim 1 or 3, wherein a proximal section of said tubular member adjacent said proximal end has a substantially constant cross-section and distal section of said tubular member adjacent said distal end has a substantially constant cross-section.

5. The system of claim 4, further comprising a transition region between said proximal and distal sections.

6. The system of claim 5, wherein said transition section comprises a funneled portion.

7. The system of claim 5, wherein said transition section comprises a stepped portion.

8. The system of claim 3, wherein said distal section has a length between about 1.75 inches and about 4 inches.

9. The system of claim 8, wherein said distal section has a length between about 2.0 inches and about 2.25 inches.

10. The system of claim 3, wherein said proximal section has a length between about 0.5 inches and about 10 inches.

11. The system of claim 4, wherein said proximal section has about external diameter at about 0.120 inches and said distal section has and external diameter at about 0.77 inches.

12. The system of claim 3, wherein said distal section has a wall thickness that is less than a wall thickness of said proximal section.

13. The system of claim 4, wherein said distal section has a wall thickness that is less than a wall thickness of said proximal section.

14. The system of claim 1 or 3, in combination with a stylet having a proximal end and a distal end.

15. The system of claim 14, further comprising a handle, said handle comprising a proximal portion associated with said stylet proximal end and a distal portion associated with said cannula proximal end.

16. The system of claim 15, wherein said handle distal portion includes a fitting for connection to an implant material driving system.

17. A cannula made by:
providing a cannula having a substantially consistent outside diameter, and
forming a distal portion of said cannula to a reduced diameter relative to a proximal portion.

18. The cannula of claim 17, wherein said forming is performed by swaging.

19. The cannula of claim 17, wherein said forming is performed by grinding.

20. The cannula of claim 19, wherein said grinding is plunge grinding.
